(19) United States
(12) Patent Application Publication
(10) Pub. No.: US 2026/0272359 A1
(43) Pub. Date: Sep. 17, 2026

(54) MEDICAL ELECTRODE AND SYSTEM THEREOF

(71) Applicant: INNACCEL TECHNOLOGIES PRIVATE LIMITED, Sampangiramanagar Bangalore Karnataka (IN)

(72) Inventors: Vibhav JOSHI, Sampangiramanagar Bangalore (IN); Sumedh KAULGUD, Sampangiramanagar Bangalore (IN); Aditya MATHUR, Sampangiramanagar Bangalore (IN); Adaikkammai S, Sampangiramanagar Bangalore (IN)

(21) Appl. No.: 19/669,977

(22) Filed: May 6, 2026

Related U.S. Application Data
(63) Continuation-in-part of application No. 17/947,050, filed on Sep. 16, 2022, now abandoned.
Application No. 17/947,050 is a continuation of application No. PCT/IN2021/050306, filed on Mar. 24, 2021.

(30) Foreign Application Priority Data
Mar. 25, 2020 (IN) 201941038749

(51) Int. Cl. A61B 5/288; A61B 5/00; A61B 5/257; A61B 5/265; A61B 5/266; A61B 5/27; A61B 5/282

(52) U.S. Cl. CPC A61B 5/288; A61B 5/257; A61B 5/265; A61B 5/266; A61B 5/27; A61B 5/282; A61B 5/6823; A61B 5/6833; A61B 5/6847; A61B 5/742; A61B 2503/02; A61B 2562/0215; A61B 2562/0217; A61B 2562/227

(57) ABSTRACT

Medical electrode devices configured to be worn for long period (e.g., more than an hour) in a moving patient without significantly decreasing electrical signal. In particular described herein are fetal monitoring electrodes comprising: an electrolyte; an absorbent material being in contact with the electrolyte, said absorbent material comprising a plurality of strands such that each strand is infused with the electrolyte; a pressing means being in contact with the absorbent material, said pressing means comprising a plurality of protrusions to push the strands of the absorbent material through the stratum corneum of a target skin area on a patient; and an electrically conductive means located adjacent to the pressing means, said electrically conductive means being in contact with the absorbent material. The instant medical electrode system achieves reduction of skin impedance at the site where the electrode is placed and generates low baseline noise, enabling accurate measurement of small or weak signals.

### CLAIM OF PRIORITY

This application claims priority as a continuation-in-part to U.S. patent application Ser. No. 17/947,050, titled “MEDICAL ELECTRODE AND SYSTEM THEREOF,” filed on Sep. 16, 2022, now U.S. Patent Application Publication No. 2023/0018091, which is a continuation of International Patent Application No. PCT/IN2021/050306, filed Mar. 24, 2021, now International Patent Publication No. WO 2021/191930, which claims the benefit of Indian Patent Application number 201941038749, filed Mar. 25, 2020, each of which is herein incorporated by reference in its entirety.

### FIELD

The present invention relates to a medical electrode and system thereof. The instant medical electrode achieves reduction of skin impedance at the site where the electrode is placed, without exfoliating the skin, and generates low baseline noise, enabling accurate measurement of small or weak signals including but not limited to fetal electrocardiogram (ECG).

### BACKGROUND

Medical Electrodes are used to detect electrical potential on the skin surface towards various health diagnoses. Medical electrodes that can effectively sense and detect small or weak electrical signals remain a need in the market. In fetal monitoring techniques, fetal heart rate monitoring is performed using fetal ECG. Surface electrodes with wet gel or solid get are used to pick up this signal. However, the acquisition of fetal ECG signal from the surface of the mother's abdomen is challenging as fetal ECG is of the amplitude of 5-20 micro Volts. Further, this signal is hidden in a complex of maternal ECG, signals contributed by uterine activity due to maternal contractions, and ambient noise. Picking up this small signal is made more challenging by the high electrical impedance of the stratum corneum—the topmost layer of skin. The stratum corneum damps down fetal ECG and leads to noise at the electrode skin interface. When measuring fetal ECG, the electrode is placed on the patient's belly. To reduce skin impedance at the site where the electrode is placed, a mildly abrasive paper tape is used to exfoliate the stratum corneum. Prior art electrodes like those used by the Monica electronic fetal monitor have electrodes with fixed gel area, available over a range of sizes, wherein each size requires a minimum number of exfoliation strokes with an abrasive material, for reducing skin associated impedance and noise signals to acceptable levels. However, this approach is skill dependent and often requires multiple attempts which cause discomfort to the patient and leaves scars. Thus, there is a need for an electrode that is able to generate low base line noise and reduce impedance caused by the skin, to detect small or weak signals, without requiring exfoliation of skin at the site where the electrode contacts the skin.

Another prior art electrode by Aspect Medical systems uses tines to part the topmost layer of skin and keep it parted. A gel electrolyte held in a sponge is depressed when the tines are pressed upon and this gel electrolyte seeps into the channels created by the tines. However, this approach is limited as the action of pressing on the tines and its connected electrical pad leads to uneven distribution of the gel and air pockets form between the skin-electrode interface and the electrical pad. The sponge is composed of multiple cells that hold the electrolyte gel. However, some of the sponge cells contain air. When the sponge is depressed, the air gets squeezed out along with the gel, creating air bubbles in the electrode system leading to uneven electrolyte distribution and high base line noise. Thus, there is a need for an electrode that evenly distributes the electrolyte, on application of pressure, without the generation of air bubbles.

Further, this system is particularly ineffective when the electrode is placed on an area of skin that lacks bony structures below e.g., abdominal skin. Pressing on the tines through the sponge into such skin leads to poor parting of the skin. Additionally, the air pockets lead to high electrical noise that masks small or weak signals including but not limited to fetal ECG. Thus, the afore system would be particularly ineffective for fetal monitoring as the electrode would be placed on maternal abdominal skin. Also, as the mother is not under anesthesia, multiple pressing attempts to get the electrode to part the skin, to measure the small fetal signals, make it uncomfortable for her. Hence, there is a need for an electrode that is able to measure signals on any part of the body, particularly on parts that lack a bony structure beneath the skin.

There is also a need for a low-cost electrode as the commercially available electrodes are expensive.

Thus, there is a need for a low-cost medical electrode which effectively reduces skin impedance at the electrode skin interface, has low baseline noise, permits even distribution of electrolyte without air bubbles, can be placed on any part of the body, and avoids exfoliation of skin prior to placement of electrodes reducing patient discomfort at the time of electrode placement.

### SUMMARY OF THE DISCLOSURE

In one embodiment the instant invention is a medical electrode comprising: an electrolyte; an absorbent material being in contact with the electrolyte, said absorbent material comprising a plurality of strands such that each strand is infused with the electrolyte; a pressing means being in contact with the absorbent material, said pressing means comprising a plurality of protrusions to push the strands of the absorbent material through the stratum corneum of a target skin area on a patient; an electrically conductive means located adjacent to the pressing means, said electrically conductive means being in contact with the absorbent material, wherein each electrolyte infused strand of the absorbent material functions as a conductive channel from the site of ingress in the stratum corneum to the electrically conductive means; and a support means for supporting the electrode and to hold the electrode in contact with a target skin area of a patient.

In one embodiment the invention is a medical electrode system comprising the at least one electrode, said at least one electrode being connected to a flexible base that removably engages with a measuring device.

In one embodiment the invention is a method of using the medical electrode or the medical electrode system comprising placing the electrode directly on the target skin area of a patient; pressing the electrode into the skin to push the electrolyte infused strands of the absorbent material through the stratum corneum; and displaying biopotential signal values detected by the electrode on a measuring device.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

FIG. 1A shows one example of an embodiment of the medical electrode of the instant invention

FIGS. 1B, 1C, and 1D illustrate the medical electrode of FIG. 1A being pressed into a target skin area

FIG. 1E depicts an embodiment being a multi-electrode system as described herein.

FIG. 2 shows an example of a collection of bristles including a mix (e.g. ration of between 3:1 and 5:1, e.g., 4:1) of straight to curved bristles as described herein.

FIG. 3 shows one example of a method of fabricating the bristles described herein.

FIGS. 4A-4C illustrate an example of a medical electrode as described herein. FIG. 4A sows a top view, FIG. 4B shows a back view and FIG. 4C shows a side view. Exemplary dimensions are indicated.

### DETAILED DESCRIPTION

The instant invention intends to address the afore stated technical and economic disadvantages by providing a medical electrode comprising: an electrolyte; an absorbent material being in contact with the electrolyte, said absorbent material comprising a plurality of strands such that each strand is infused with the electrolyte; a pressing means being in contact with the absorbent material, said pressing means comprising a plurality of protrusions to push the strands of the absorbent material through the stratum corneum of a target skin area on a patient; an electrically conductive means located adjacent to the pressing means, said electrically conductive means being in contact with the absorbent material, wherein each electrolyte infused strand of the absorbent material functions as a conductive channel from the site of ingress in the stratum corneum to the electrically conductive means; and a support means for supporting the electrode and to hold the electrode in contact with a target skin area of a patient.

The instant electrode creates micro perforations in the topmost dead skin layer (the stratum corneum) of the skin when pressure is applied via the pressing means. The pressing action causes the strands of the gauze to be pushed into these perforations. Further, minimal pressure application is required as the gauze is thin and easily penetrates through the skin and sticks to the ingress sites. The strands being pushed into these perforations also eliminates the need for the preparatory exfoliation step at the site at which the electrode is placed. The gel infused strands make contact with the more conductive layers of the skin underneath the stratum corneum. The gel infused strands form electrically conductive channels between the sites of ingress of the stratum corneum and the electrically conductive means of the instant electrode. Since the gel is infused in the gauze strand, it does not spread unevenly or have air bubbles which disrupt the signal transmission. In this manner, the instant electrode allows for the reduction of skin impedance, without exfoliating the skin, and generates low baseline noise leading to the accurate acquisition of weak signals including but not limited to fetal ECG.

The electrolyte gel which is in contact with the absorbent material is the electrolytic medium which allows ionic exchange between the skin surface and the electrically conductive means. The electrolyte is the medium through which biopotential measurement occurs. In one embodiment the electrolyte gel is liquid, slightly tacky or sticky as it is alginate based, highly fluid and a skin-friendly composition. The liquid nature and stickiness ensure complete absorption of the electrolyte by the gauze and enables even spreading when pressure is applied, avoiding air bubble formation.

In one embodiment of the medical electrode the absorbent material comprises the plurality of strands in a network, preferably in the gauze structure. In a preferred embodiment of the medical electrode the absorbent material is of cotton fibers. In the prior art sponge is the absorbent material used in the medical electrodes, said sponge is composed of multiple cells which hold the electrolyte gel. When the sponge is pressed, the gel in the cells is squeezed out. However, electrolyte distribution is often uneven as some of the sponge cells have trapped air and air bubbles are introduced into the system. These air bubbles are to be avoided as they cause high base line noise. Instead, the instant invention uses cotton fibers arranged in the gauze structure. The electrolyte gel gets absorbed into the individual strands of the gauze and does not get squeezed out when the strands are pressed on, the cotton strand itself becomes a conductive channel.

In one embodiment of the medical electrode the pressing means is a plastic micro-bristle structure in contact with a plurality of strands of the absorbent material. In another embodiment the pressing means has a further means on a top surface of the electrode to aid application of pressure on the electrode, said top surface being the surface facing away from the target skin area. The said further means being an external mechanical or electromechanical means of applying pressure onto the electrode. Said further means applies a range of pressure for a specific amount of time due to physical, electrical or chemical characteristics. In one embodiment the further means is a bubble on top of the electrode with a standard amount of air inside the bubble. A specific amount/range of pressure is required to be applied to burst it. Till the bubble is burst, the pressure built up inside is applied evenly across the bottom surface of the bubble, which is aligned with the pressing means on the top surface of the medical electrode, said surface being the one facing away from the skin. In another embodiment the further means is a plastic disk designed to crack once a specific amount of pressure is reached, up to that point it distributes the pressure evenly.

In one embodiment of the medical electrode the electrically conductive means is located adjacent to the pressing means. The term adjacent in this context includes the electrically conductive means being placed near, next to, by the side of, on top of, or offset to the pressing means. In one embodiment of the medical electrode the electrically conductive means is a plastic substrate coated with a conductive metallic layer on the surface in contact with the absorbent material, said surface facing the target skin area. In one embodiment of the medical electrode the conductive metallic layer is of silver/silver chloride. In one embodiment of the medical electrode the plastic substrate is electrically attached to a flexible printed circuit board. In one embodiment the electrically conductive means has a stainless-steel component on the top surface which interfaces/locks into standard electrocardiogram cable connectors to form a connection with any measuring device including but not limited to a fetal or adult ECG device, neural signal measurement device (e.g., EEG) etc. The stainless-steel component includes but is not limited to a stud, a wire, crocodile clip connector, banana stud connector, or a universal snap and tab connector.

In one embodiment the medical electrode is connected to a measuring device for measuring and recording biopotential signals detected by the electrode. This electrode can be utilized for measuring any biopotential signals including but not limited to fetal and adult ECG measurement, neural signal measurement etc. The instant invention is particularly designed to detect and measure small or weak signals through the skin as it reduces skin impedance and generates low baseline noise at the site of ingress of the instant electrode, without requiring exfoliation of the skin at the site of electrode contact.

In one embodiment the support means is for supporting the electrode and to hold the electrode in contact with a target skin area of a patient. In one embodiment the support means comprises two tapes, a backing tape on which is assembled the electrode components and a foam-based pressure sensitive adhesive for adhering the assembled electrode to the skin of the patient. In another embodiment the support means further consists of a covering being a sheet, a film, or a membrane with liquid resistant characteristics on the top surface of the electrode, said top surface being away from the skin of the patient.

In one embodiment the medical electrode comprises a medical electrode system comprising at least one electrode, said at least one electrode being connected to a flexible base that removably engages with a measuring device. The at least one electrode system comprises more than one electrode being connected to a flexible base to create a multi-electrode system, said multi-electrode system being connected to a measuring device.

In one embodiment it is a method of using the medical electrode or the medical electrode system comprising more than one electrode, said method comprising placing the electrode directly on the target skin area of a patient; pressing the electrode into the skin to push the electrolyte infused strands of the absorbent material through the stratum corneum; and displaying biopotential signal values detected by the electrode on a measuring device.

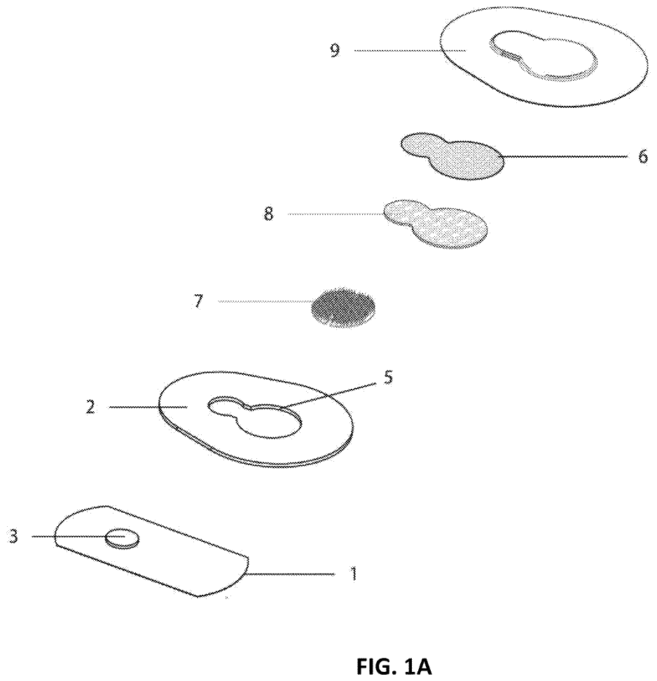

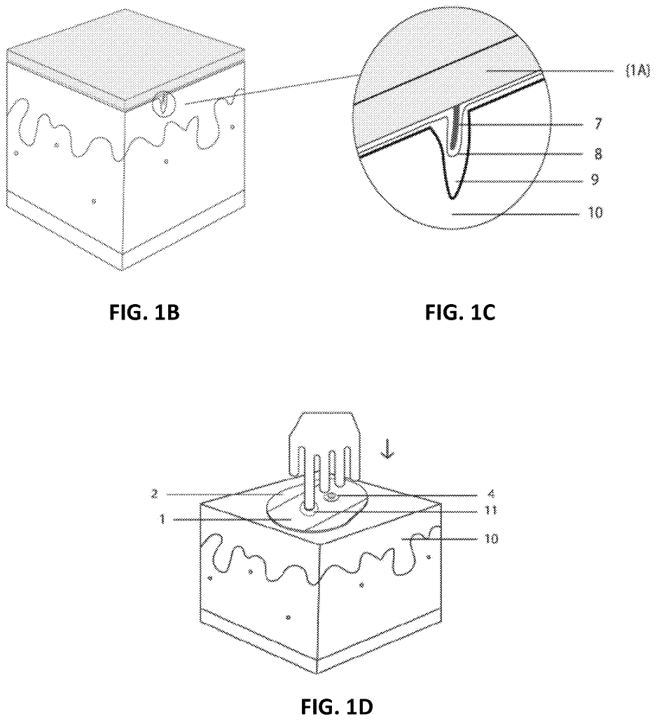

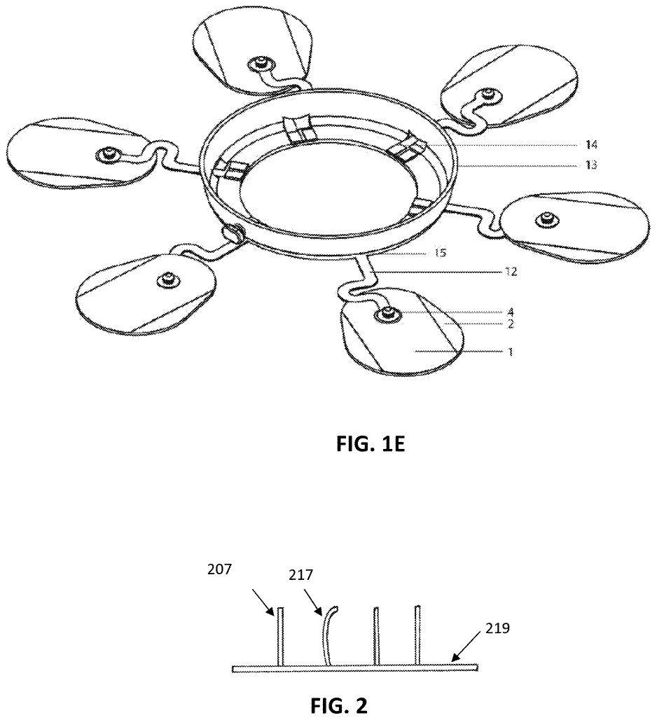

FIG. 1A shows an embodiment of the medical electrode of the instant invention. The figure depicts the medical electrode (100) in its disassembled form, comprising a single layer or multiple layers of soft cotton in gauze or gauze-like structure (8) infused with electrolyte (6), a pressing means (7) in contact with the cotton gauze (8) (in the assembled form) for pressing the strands of gauze into the skin at multiple locations, and an electrically conductive means (3). The pressing means (7) in this embodiment comprises a plastic micro-bristle structure with vertical plastic bristles. The strands of the gauze (8) form electrical channels between the site of the ingress (9) into the stratum corneum (10) and the electrically conductive means (3) (FIG. 1C). The gauze (8), electrically conductive means (3) and pressing means (7) are assembled on a backing tape (1) enclosed in a foam-based pressure-sensitive adhesive (2). The cavity (5) in the pressure sensitive adhesive is shaped so as to concentrate the gauze strands at the juncture with the electrically conductive means (3). In this embodiment the electrically conductive means (3) is offset from the pressing means (7), so pressure is applied directly over the gauze (8).

Assembly of the electrode involves placing the electrically conductive means (3) on the adhesive side of the backing tape (1), said adhesive side facing towards the patient's skin. The pressing means (7) with the micro bristles is stuck offset from the electrically conductive means (3) onto this adhesive side with the bristles pointing towards the skin side. This structure is now assembled into a cavity (5) in a foam-based pressure-sensitive adhesive (2) so that the adhesive side of the pressure-sensitive adhesive is facing towards the skin. The cavity is closed by the backing tape so the system is closed from the top side, the surface not facing the skin. The gauze (8) is placed in cavity (5) to occupy the remaining volume of the cavity and the electrolyte gel (6) is added. The gauze absorbs the electrolyte gel. The gel is the electrolytic medium which allows ionic exchange between the skin surface and the electrically conductive means coated with silver/silver chloride. The electrolyte is the medium through which the biopotential measurement occurs.

FIGS. 1B, 1C and 1D depict the medical electrode (100) of FIG. 1A being pressed into a target skin area. The plastic bristles of the pressing means (7) push the strands of the absorbent material (8) into the stratum corneum (10) at the site of ingress (9). FIG. 1D shows when pressure is applied on the pressing means (arrow depicts the direction of pressure) to push the bristles, hence the electrolyte infused strands of the absorbent material, into the stratum corneum (10). The electrode has a stainless-steel component (4) on the top surface which interfaces/locks into standard electrocardiogram cable connectors to form a connection with any measuring device. The electrode in this embodiment has a further means (11) on the top surface to aid application of pressure on the electrode.

FIG. 1E depicts an embodiment being a multi-electrode system. In this embodiment the multi-electrode patch comprises six electrodes. The electrodes are connected to a flexible base, for removably engaging with a monitoring device for detecting a maternal and/or fetal electrophysiological signal from the electrodes. The flexible base in this embodiment comprises a flexible substrate (12), a plastic unit (13) and a pressure-sensitive adhesive foam ring (15) for attaching the base of the multi-electrode patch to the skin of the patient. The module has a mechanism for removable mechanical engagement with the monitoring device, and an electrical connection unit (14) for making an electrical connection from the electrodes to the readout device. Engaging the patch with the monitoring device comprises both the mechanical module unit and the electrical module unit.

In general, the electrodes described herein are configured to be worn for longer than contemporary electrodes, including tissue-penetrating electrodes, and in particular are adapted for use for fetal monitoring in a pregnant patient. For example, as described above, these apparatuses may be configured for multi-hour ambulatory fetal monitoring electrodes and may be specifically adapted for this use.

The maternal abdomen is typically a compliant tissue. Skin, subcutaneous fat, rectus sheath, and gravid uterus form a multilayer compliant target with no underlying bony structure. Prior art press-and-release mechanisms that rely on a firm reactive surface cannot maintain reproducible electrode-skin contact on soft tissue such as the abdomen. Such soft tissue absorbs the force used to press the device, and any electrolyte channel formed at application typically collapses as the press force decays, and with patient (e.g., maternal) movement. Further, unlike many other indications, fetal monitoring is multi-hour and ambulatory. Labor monitoring typically runs for hours including ambulation, position change, and uterine contractions. Capturing fetal ECG under maternal motion requires that the bristle-formed ingress sites are maintained, not merely initially formed.

As a consequence, the devices and systems (e.g., apparatuses, including medical electrodes) described herein are particularly well adapted to allow long (e.g., greater than 30 minutes, >45 minutes, >1 hour, >1.5 hours, >2 hours, >2.5 hours, >3 hours, etc.) monitoring, without significantly declining in signal. For example, these apparatuses may be adapted to remain seated and continue to press the cotton-gauze strands into the stratum corneum throughout monitoring over these long periods. This may be achieved, at least in part, by the use of a tensile-rated backing tape as the support, a die-cut bristle disk (e.g., forming the micro-bristle structure) having a mixed straight and partial-hook population of micro-bristles, and a perimeter retention notch. These apparatuses may include a prolonged-wear gel composition, and a woven cotton gauze whose thread geometry is co-engineered with the bristle array to deliver multi-location ingress.

For example, as described above, the support may be a backing tape. In some cases the backing tape may be selected to have a tensile strength (lengthwise at break) of between about 2-4 kgf/24 mm (e.g., about 3.0±0.5 kgf/24 mm, as measured by, e.g., an ASTM-style tensile machine, COA Sl. 12). This range of tensile strength may be particularly helpful to resist in-plane stretch under cable tug and maternal motion and may keep the bristle disk pressed against the gauze and stratum corneum without displacement. The support (e.g., backing tape) may have a tensile in elongation (e.g., lengthwise at break) of greater than about 300% (e.g., COA Sl. 13); this range of tensile elongation may allow the support to recover without tearing under repeated flexion during labor. Any of these supports may have a peel adhesion (ASTM D 903) of between about 1-3 kgf/24 mm (e.g., about 2.0±0.5 kgf/24 mm), which may help sustain skin bond for prolonged wear. The support may be any appropriate material, including, for example, a carrier film such as a Metallocene polyethylene (e.g., about 100±5 μm). Metallocene polyethylene (mPE) materials typically have a narrower MWD than Ziegler-Natta polyethylene (ZN-PE), providing a uniform peel and conformability that may be particularly useful.

The support may have a total laminate thickness of between about 140 to 180 μm (e.g., 160±5 μm) and may flex with abdominal contour without lifting edges. In any of these methods and apparatuses the support may be treated, e.g., adhesive/surface prep, by a hot melt of 30-35 g/m²; corona 42 dyne, which may provide a constant-contact PSA. Corona treatment may promote adhesive anchorage to the carrier film.

The combination of lengthwise tensile strength (~3 kgf/24 mm), tensile elongation (>300%), and peel adhesion (~2 kgf/24 mm) may be selected to hold the bristle array pressed into the gauze and the gauze pressed into the stratum corneum during body (e.g., maternal) motion. Tensile strength is the load-bearing parameter; under cable tug and maternal motion, a support with insufficient tensile may stretch in-plane and allow the bristle disk to shift relative to the skin, breaking gauze-skin contact.

In general, the tensile strength specified above (tensile of about 3.0±0.5 kgf per 24 mm strip width) may correspond to ~1.22 N/mm of tensile capacity per unit width. This parameter may be scaled to the support dimensions. For example, scaled to a 28 mm backing-paper width, the tensile capacity may be approximately 3.5 kgf (~34 N) at break; across the 58.36 mm overall assembly length, approximately 7.3 kgf (~72 N) at break. Equivalent break stress over the 24 mm×0.160 mm laminate cross-section is approximately 7.7 MPa.

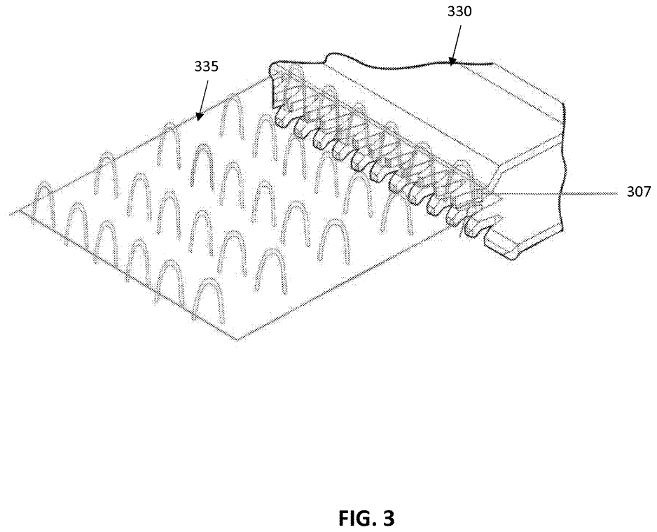

As mentioned above, the press may include plastic (e.g., non-electrically conductive/electrically insulative) micro-bristles. In some cases the press may be configured as a bristle disk (e.g., formed of Nylon 66, die-cut). For example, the micro-bristles may be formed of a polymer such as Nylon 66 (PA66), which may be die-cut from nylon Velcro hook stock to a disk (e.g., having a diameter of between about 10-50 mm, e.g., about 18.4±0.2 mm disk). This may help stablish skin-contact. Bristles may be formed by cutting specifically to generate a mixed bristle population as described below and shown in FIGS. 2 and 3.

In any of these apparatuses, it has been found that a mixed bristle population having a majority of straight and a minority of curved bristles (e.g., between 51%-95% straight/5%-49% curved, 60-80% straight/20%-40% curved, ≈70% straight protrusions/≈30% curved) may be particularly advantageous. As used herein “curved” may refer to bristles retaining a small lateral hook 217 (between 10 degrees and 60 degrees), whereas straight bristles have less than 10 degree curvature at their distal end region. Straight bristles may press cotton-gauze strands into the stratum corneum, whereas partial-hook bristles provide surface micro-engagement (“micro-pull”) that anchors the array against lateral skin shear during maternal motion. By tuning the amount of straight versus curved bristles, as described herein, the ability to make and maintain the long-lasting (e.g., >30 min) contact even while the patient is moving, may be surprisingly effective as compared systems with too many or not enough curved bristles. Both straight and curved bristles may be referred to as “vertical” bristles. FIG. 2 show an example of a portion of a press 219 including a mixture of straight 207 and curved 217 bristles.

Any of the presses including bristles described herein may also or alternatively include a retention notch (e.g., a disk retention notch) that is formed into the perimeter of the press; this retention notch may lock the bristles (e.g., the bristle disk) against rotational/translational displacement within the support assembly under motion. For example, a retention notch (e.g., a disk retention notch) may be about 2.50 mm×1.50 mm and may be die-cut into the disk perimeter.

The dimension of the bristles may be selected to penetrate through a stratum corneum of the target skin (e.g., sort tissue, and in particular the material abdominal skin). For example the bristles may have a height (above the base of the press) of between about 0.7 mm and 1.4 mm (e.g., between about 0.8 mm and about 1.2 mm, between about 0.9 mm and 1.1 mm, etc.). The bristles may have a diameter of about 0.1 mm-0.2 mm (e.g., about 0.15 mm). These dimensions may be configured for ingress through the absorbent material (e.g., cotton gauze, such as 0.30 mm gauze) layer into superficial skin.

The bristles may be arranged in any appropriate density, such as, for example, between about 100 bristles/cm² and 150 bristles/cm² (e.g., between about 110 bristles/cm² and 135 bristles/cm², about 122-124 bristles/cm², etc.). The bristles may be arranged at a pitch of about 0.80 mm to 1 mm (e.g., pitch of about 0.90 mm). The bristles may be distributed over the press. In some examples the pitch may be co-engineered with gauze thread pitch (e.g., between about 0.8-1.0 mm). The bristles may have a flexural modulus of between about 2.2 and 3.4 GPa (e.g., ≈2.8 GPa) and a yield strain of between about 3%-6%) (e.g., ≈4-5%, Rockwell M≈80. This modulus may provide a stiffness for press without buckling; elastic recovery across repeated cycles; hardness for stratum-corneum indent without skin laceration. In general, the bristles may be stable in mild-aqueous contact at pH≈6 (ISO 10993-5/-10 use in skin-contact devices) and may be used for prolonged skin apposition.

Thus, in any of these apparatuses, the bristle disk may be die-cut (using a tool 330 for cutting) from a material such as a Nylon 66 Velcro hook stock 335. This is illustrated in FIG. 3, for example. The material may be cut from loops 307 (e.g., having a loop length of about 1.8 mm in FIG. 3). The source material may be processed (e.g., cut, heated, etc.) to form a mixed population of straight/curved bristles, as described above. For example, the bristle population may be approximately 70% straight protrusions, which may press cotton-gauze strands into the stratum corneum, and approximately 30% retaining a small lateral hook, which produces a surface micro-engagement (“micro-pull”) that anchors the array against lateral skin shear during maternal motion, as described above. A retention notch cut into the perimeter of the press may further lock the press against rotational/translational displacement within the support. In combination with the properties of support (e.g., backing tape) such as the tensile properties (~3 kgf/24 mm), peel (~2 kgf/24 mm), and >300% elongation described above, these features together may help maintain gauze-stratum-corneum contact during continuous monitoring under maternal motion.

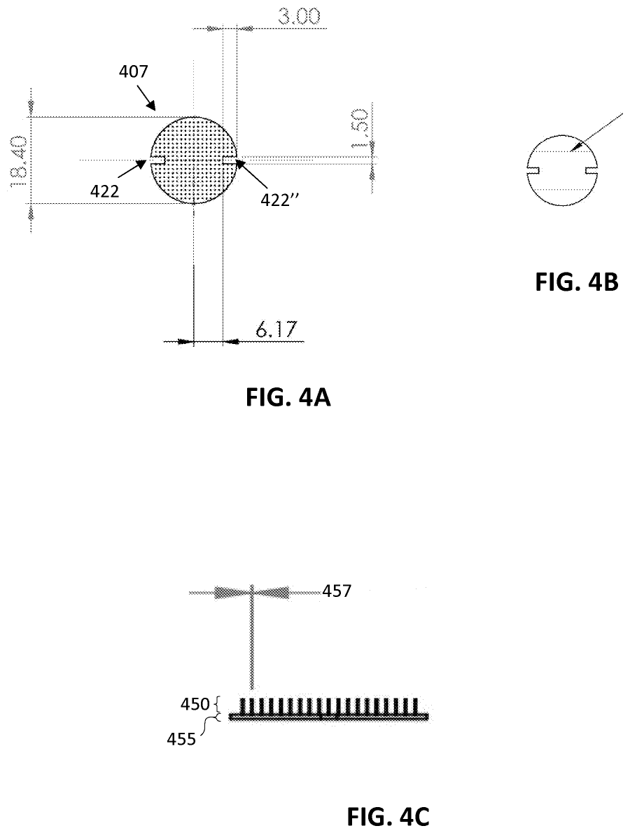

FIGS. 4A-4C show another example of a portion of a medical electrode configured for fetal monitoring as also shown in FIG. 1, including, in FIG. 4A, a press 407 having a pair of notches 422, 422′ (exemplary dimensions are shown). FIG. 4B shows an example of a back of the press. FIG. 4C shows an enlarged view of one example of a set of micro-bristles (which may include a mixed population of straight/curved bristles) having an average height 450 of, e.g., between 1.2 mm-1.6 mm, extending from a base having a thickness of about 0.4 mm. These bristles are shown having a diameter of about 0.15 mm.

An appropriate electrolyte may be, for example, an electrolyte gel composition which may be selected for prolonged wear, as described herein. For example, the electrolyte may be a gel matrix of, e.g., sodium alginate (5%) configured to provide a cohesive hydrogel reservoir. The electrolyte gel may include a conductive salt, such as, e.g., sodium chloride (6%), which is above isotonic (~0.9% NaCl), and may support ionic current density for fetal-ECG capture through maternal tissue. Any of these electrolyte compositions may be configured for prolonged wear and may include, for example, a humectant/anti-dry agent such as propylene glycol (e.g., 10%), PEG (3%), Sodium PCA (0.5%), this may limit dehydration of the gel during multi-hour wear. Any of these electrolyte gels may include skin protection and/or anti-irritant compositions such as one or more of: Hyaluronic acid (e.g., 1%), Aloe vera gel (e.g., 1%), Dimethicone (e.g., 350), cSt (e.g., 1.5%), Light liquid paraffin (e.g., 1.5%), Calamine (e.g., 0.5%), Glycerol monostearate (e.g., 5%), etc. These compounds may be configured as part of a skin-protection envelope intended for tolerance of high ionic load and continuous bristle press. In addition any of these electrode material may include a topical anesthetic, such as, e.g., Pramoxine HCl (e.g., 0.1%), that may reduce sensation associated with sustained pressing. Any of these compositions may also or alternatively includes an antimicrobial/preservative (e.g., one or more of: antifungal (e.g., 7%), Phenoxyethanol (e.g., 1%), NNIB (e.g., 0.03%), Vitamin E, etc.), which may preserves the gel-skin interface during prolonged wear.

The electrolyte gel may be formulated to have a conductivity (e.g., with about 6% NaCl) to be well above isotonic, which may supports ionic current density at the gauze-skin interface; the humectant and occlusive components may also be selected to limit the impedance rise that would otherwise follow gel dehydration. In addition, any of these electrolyte gels may be configured to have a skin tolerance for prolonged wear such that the combined skin-protection components (e.g., hyaluronic acid, aloe, calamine, dimethicone, light liquid paraffin, glycerol monostearate), the topical anesthetic (e.g., pramoxine HCl), and the antimicrobial/preservative system (e.g., antifungal, phenoxyethanol, NNIB, Vitamin E) are directed to tolerance of continuous bristle press and the warm, occluded contact zone over a labor-length monitoring session.

The absorbent material may be a cotton gauze (e.g., woven structure co-engineered with the bristle array). This may be 100% woven cotton and die-cut from a 100×10⁰ mm 24-ply sheet. The gauze may be hydrophilic absorbent; retains alginate gel of CC3 under compression. The gauze may have a thread density of about 7×10 threads/cm² (warp×weft), and may have an ordered, predictable strand layout, in which each strand is a candidate conductive channel. The inter-thread spacing may be, e.g., between about 0.8-1.0 mm, which may be co-engineered with bristle pitch (0.90 mm) of CC2, so that each bristle approximately aligns with a discrete gauze thread intersection. For example, single thread thickness may be about 0.10 mm, and each strand may be individually drivable through the stratum corneum by an aligned bristle. The gauze may have a stack configuration of 2 or more (e.g., 3 or more) layers, and a total stack thickness of about 0.30 mm. This may provide a multi-layer reservoir for gel, with multiple strand layers per ingress site. The active footprint may be, e.g., about 32.06×21.8 mm; 384.20 mm² covered surface area, which may spread ingress sites across an extended skin area beneath the bristle disk. Thus, in some examples more than 200 bristles may be engaged per disk (e.g., ≈325 total bristles within the 18.4 mm disk) so about 225 straight and about 95 partial-hook. The multi-location ingress may include hundreds of simultaneous conductive channels through the stratum corneum per electrode, distributed across the disk footprint rather than concentrated at one location

Thus, this configuration of mesh may allow multi-location ingress. The thread density (e.g., greater than 1×10/cm², e.g., 7×10/cm²) and inter-thread spacing (e.g., 0.8-1.0 mm) of the gauze may be co-engineered with the bristle density (e.g., 122-124/cm²) and pitch (e.g., 0.90 mm). Across the press (e.g., an 18.4 mm bristle disk), this produces approximately 325 total bristles (≈225 straight bristles), each aligned with a discrete gauze thread intersection and pressing the strand into the stratum corneum, and about 95 partial-hook bristles distributed across the disk providing surface micro-engagement. The result is multi-location ingress: hundreds of simultaneous conductive channels through the stratum corneum at each electrode, spread across the disk footprint rather than concentrated at a single location. This distributed character may make the array tolerant to motion; while individual ingress sites momentarily lose contact during a movement, the population as a whole remains in conductive contact, and fetal-ECG capture is sustained.

The following experimental example is illustrative of the invention but not limitative of the scope thereof:

### Working Example 1

The noise characteristics of electrodes of the instant invention versus the prior art electrodes was performed. The instant electrodes comprise an absorbent material being cotton gauze while the prior art electrodes comprise the sponge-based systems. The two electrodes were applied on different locations on the abdomen at a distance of 6 cm from each other. The noise at the sites was measured using 3M solid gel electrodes and was found to be equivalent. These values were used as a base-line of the noise level prior to application of the sponge or gauze electrode.

The study sampled 40 subjects of different ages, gender, and skin types. The results of 5 scans are shown in the table below. The noise detected by the cotton gauze electrode is at least 3-fold lower than the sponge-based electrode, with subject 2 showing a 20-fold reduction in noise.

| | Gauze | Sponge | | |
|---|---|---|---|---|
| | electrode site | electrode site | | |
| | control noise | control noise | Gauze | |
| Subject | (3 M solid gel | (3 M solid gel | Electrode | Sponge Electrode |
| no | electrodes) | electrodes) | Noise | Noise |
| 1 | 60 microV | 53 microV | 5 microV | 17 microV |
| 2 | 48 microV | 48 microV | 1.2 microV | 22 microV |
| 3 | 85 microV | 83 microV | 3.5 microV | 24 microV |
| 4 | 24 microV | 29 microV | 1.5 microV | 18 microV |
| 5 | 47 microV | 52 microV | 4 microV | 20 microV |

Thus, the instant invention comprises a low-cost medical electrode that effectively reduces skin impedance at the electrode skin interface, has low base line noise signals, avoids exfoliation of skin prior to placement of electrodes, permits even distribution of electrolyte without air bubbles, can be placed on any part of the body, and reduces patient discomfort at the time of electrode placement.

While the present invention has been described with respect to certain embodiments, it will be apparent to those skilled in the art that various changes and modifications may be made without departing from the scope of the invention as defined in the following claims.

What is claimed is:

1. A medical electrode device configured for long-term fetal monitoring from an abdomen of a pregnant woman, the device comprising:
an absorbent material infused with an electrolyte gel, wherein the absorbent material comprises a cotton gauze having a plurality of strands wherein each strand is infused with the electrolyte gel;
a press in contact with the absorbent material, the press comprising a plastic micro-bristle structure with a plurality of vertical plastic bristles sized to push the plurality of strands through a stratum corneum of a target skin area on a patient and to form channels that include the plurality of electrolyte infused strands within the stratum corneum;
an electrically conductive substrate adjacent to the press, the electrically conductive substrate in electrical contact with the absorbent material, wherein each electrolyte infused strand of the absorbent material is configured to function as a conductive channel from a site of ingress in the stratum corneum to the electrically conductive substrate; and
a support configured to hold the medical electrode in contact with the target skin area.

2. The device of claim 1, wherein the plastic bristles comprises a mixed population of between 60-80% straight bristles and between 20-40% curved bristles.

3. The device of claim 1, wherein the bristles are between 0.7 and 1.4 mm high.

4. The device of claim 1, wherein the strands are arranged in a mesh so that the bristles are configured to push a single strand through the stratum corneum.

5. The device of claim 1, wherein the electrically conductive substrate is a plastic substrate coated with a conductive metallic layer on a surface that is in contact with the absorbent material and is configured to face the target skin area.

6. The device of claim 5, wherein the conductive metallic layer is silver/silver chloride.

7. The device of claim 1, wherein a top surface of the press is configured to distribute applied pressure on the medical electrode.

8. The device of claim 1, wherein the support comprises a foam material comprising a pressure-sensitive adhesive surrounding the absorbent material.

9. The device of claim 1, wherein the electrically conductive substrate is in electrical contact with the absorbent material through a layer of electrolyte gel on the electrically conductive substrate.

10. The device of claim 1, further comprising an electrical contact in electrical communication with the electrically conductive substrate that is configured to couple to an electrical cable.

11. The device of claim 1, further comprising a flexible base configured to removably engage with a measuring device.

12. The device of claim 1, wherein the electrically conductive substrate is offset from the press and the absorbent material so that pressure applied by the press is applied directly over the absorbent material.

13. The device of claim 1, wherein the support comprises a flexible base configured to engage with a fetal monitor.

14. A medical electrode device configured for long-term fetal monitoring from an abdomen of a pregnant woman, the device comprising:
an absorbent material infused with an electrolyte gel, wherein the absorbent material comprises a cotton gauze having a plurality of strands wherein each strand is infused with the electrolyte gel;
a press in contact with the absorbent material, the press comprising a plastic micro-bristle structure with a plurality of vertical plastic bristles, comprising a mixed population of between 60-80% straight and 20-40% curved bristles, wherein the bristles are sized to push the plurality of strands through a stratum corneum of a target skin area on a patient and to form channels that include the plurality of electrolyte infused strands within the stratum corneum; and
an electrically conductive substrate adjacent to the press, the electrically conductive substrate in electrical contact with the absorbent material, wherein each electrolyte infused strand of the absorbent material is configured to function as a conductive channel from a site of ingress in the stratum corneum to the electrically conductive substrate.

15. A method of fetal monitoring, the method comprising:
placing a plurality of medical electrodes coupled to a fetal monitor on a target skin area of a pregnant patient's abdomen;
pressing on a press of each of the medical electrode, the press comprising a plastic micro-bristle structure with a plurality of vertical plastic bristles, wherein pressing on the press causes the plurality of vertical plastic bristles to drive a plurality of strands of an absorbent material through a stratum corneum of the target skin area, wherein each strand of the plurality of strands is infused with an electrolyte gel, wherein the plurality of strands are formed of cotton gauze;
forming an electrically conductive channel, which includes the plurality of strands infused with the electrolyte gel, from a site of ingress in the stratum corneum to an electrically conductive substrate of the medical electrode through the plurality of strands; and
detecting a signal from the electrically conductive substrate.

16. The method of claim 15, wherein placing the medical electrode on the target skin area comprises adhesively securing the medical electrode to the target skin area.

17. The method of claim 15, wherein placing the medical electrode on the target skin area comprises placing the medical electrode without exfoliating the target skin area first.

18. The method of claim 15, further comprising displaying the signal detected on a measuring device.
